(19) United States
(12) Patent Application Publication
(10) Pub. No.: US 2022/0387813 A1
(43) Pub. Date: Dec. 8, 2022

(54) Light based body contouring system

(71) Applicant: Robert Mc Elyea, (US)

(72) Inventor: Robert Mc Elyea, (US)

(21) Appl. No.: 17/340,022

(22) Filed: Jun. 6, 2021

(51) Int. Cl. A61N 5/06

(52) U.S. Cl. CPC A61N 5/06; A61N 2005/0658; A61N 2005/0651; A61N 2005/0643

(57) ABSTRACT

A noninvasive method of reducing a user's body apparent body fat by applying a device emitting high power light having a wavelength shorter than 1065 nm externally through the skin of the patient. The device is placed on or attached to one or more areas of a patient's body. Sufficient energy, with a targeted value of 6.6 J/cm absorbed, is applied to one or more of those areas to cause a reduction in the measurement in the energized areas. The preferred methodology uses LEDs at about 532 nm and 635 nm. Targeted energy levels are a minimum of 2 W/cm for LED emissions applied every other day for a minimum of 75 seconds per exposed area; until desired amount of size reduction is achieved.

### FIELD OF INVENTION

This invention relates to a method for non-invasive method of shaping and contouring a human body. In particular, this invention relates to the application of light with wavelengths shorter than 1065 nm to targeted external regions of a patient's body to slim the patient's body.

### BACKGROUND

There is an obesity epidemic in the world with many people having difficulties eating the optimal amount of calories. Combined with the societal drive to be slim and the concurrent potential health issues with obesity there is a desire by many people to appear slimmer. This has resulted in a proliferation in the market place for multiple methodologies to reduce body size and apparent body fat. Many people despite healthy diets and an exercise routine still have stubborn areas of residual fat that require a targeted method of reduction. And due to high costs as well as issues with body confidence many people are resistant to seek this contouring via traditional methods of cosmetic surgeries.

This reluctance to invasive methods or surgery have led to a wide variety of noninvasive methods such as electromagnetic energy, such as microwave, ultrasound or radio frequency radiation, has been used to reduce fat. Multiple U.S. Pat. Nos. 5,507,790; 5,143,063 use destructive methods to reduce fat with an associated recovery time and risk. Additionally multiple noninvasive methods using laser light alone i.e. 2005/0203594 to release at least a portion of the intracellular fat into the interstitial spaces were created. This method revolutionized the market for body contouring because it reduced fat with no trauma to the patient.

While all of the methods above are effective they have the end result of not being available for home use. This invention offers the ability to safely, efficiently, and effectively provide the capabilities of this outpatient medical procedures to be conducted in the safety and privacy of the user's residence with minimal risks to the patient.

### SUMMARY OF THE INVENTION

This invention is a noninvasive method of slimming a patient's body by applying a mix of high power light having a wavelength shorter than 1065 nm externally through the skin of the patient. The device is placed on or attached to one or more areas of a patient's body. Sufficient energy, with a targeted value of 6.6 J/cm absorbed, is applied to one or more of those areas to cause a reduction in the measurement in the energized areas. The preferred methodology uses LEDs at about 532 nm and 635 nm. Targeted energy levels are a minimum of 2 W/cm for LED emissions applied every other day for a minimum of 75 seconds per exposed area; until desired amount of size reduction is achieved

### DETAILED DESCRIPTION OF THE INVENTION

Multiple LEDs of 532 and 635 nm emit light waves which can be described by frequency, wavelength, or energy. These are encapsulated into a device which is either placed upon or attached to the user's body.

In the method's simplest form, a user's body is measured initially, light therapy is applied to the user, and the measurement is repeated shortly after laser treatment to determine whether the measurement is reduced.

Measurements are made using objective, non-invasive modes to determine the user's external dimensions with tape measures, being the preferred method. Using external dimension as the measurement mode in a first example, an initial measurement is made of the circumference of one or more areas of a user's body. For example, a tape measure is used to measure the circumference of the user's torso at the navel. Light energy of a wavelength less than about 1065 nm is applied to the user at and around the navel. Sufficient light energy is applied to the user to cause the torso measurement to be reduced, typically about 6.6 J/cm.

A delay of 48 hours is executed to enable the body's natural processes to clear the system of released fatty acids, triglycerides, glycol, and other substances and a second measurement is taken. This enables the user to establish a sense of self confidence and certitude of the invention's function.

The process is repeated until the measurement is less than the initial measurement or the user's desired level of circumference reduction is achieved.

The dosage of light energy required to achieve slimming will vary depending on the thickness of the user's skin, thickness of fatty tissue, and other biological factors peculiar to each user.

We claim:

1. A method of slimming a patient, the method comprising: 1) making an initial measurement of a first area of a patient's body to determine circumference, 2) applying sufficient light of a wavelength of less than 1065 nm to the first area of the patient's body, 3) letting the body's natural processes function for 0.48 hours to respond to the exposure, 4) making a second measurement of the exposed areas of the patient's body; and 5) comparing the measurements. Repeating the process until the desired reductions in patient's size is achieved.
